(19) United States
(12) Patent Application Publication
(10) Pub. No.: US 2025/0382581 A1
(43) Pub. Date: Dec. 18, 2025

(54) CELL CULTURE DEVICE AND USES THEREOF

(71) Applicants: UNIVERSITETET I OSLO, Oslo (NO); OSLO UNIVERSITETSSYKEHUS HF, Oslo (NO)

(72) Inventors: Henry W. HOYLE, Oslo (NO); Anna Katharina FRANK, Fjellstrand (NO); Espen MELUM, Oslo (NO); Stefan KRAUSS, Eidsvoll Verk (NO)

(21) Appl. No.: 18/746,212

(22) Filed: Jun. 18, 2024

(51) Int. Cl. C12N 5/071; C12M 1/00; C12M 1/12; C12M 3/00; C12M 3/06

(52) U.S. Cl. CPC C12N 5/0691; C12M 21/08; C12M 23/16; C12M 25/10; C12M 29/10; C12N 2513/00

(57) ABSTRACT

A cell culture device includes a well plate and a cover sheet. The well plate includes at least one culture arrangement, the culture arrangement including a row of wells having a first outer well, a central well and a second outer well. An access port provides an opening into the first outer well. A passage (e.g., a straight or linear passage) extends from the access port to each of the first outer well, central well and second outer well. The passage passing through a first hole in a first well wall separating the first outer well and the central well. The passage passing through a second hole in a second well wall separating the central well and second outer well. The cover sheet is provided on an underside of the well plate.

### TECHNICAL FIELD

The disclosure relates to a cell culture device, particularly, to a cell culture device for culturing epithelial cells in apical-basal environments.

### BACKGROUND

The convergence of lab-on-a-chip (LOC) technology and cell biology has permitted the study of human physiology in an organ-specific context in so-called organ-on-a-chip (OOC) devices. These devices allow for the growth of cells in complex 3D morphologies using cell scaffolding promoting higher levels of cell differentiation and tissue organization far exceeding the possibilities of traditional 2D culture systems (e.g., plate culture systems).

However, established 3D culture models still struggle to mimic organs or tissues with complex cell structures and organisation as these models fail to recreate all the different microvariant properties such as tissue-to-tissue interfaces, the chemical and pH gradients occurring on a cellular scale, and micro-mechanical forces (e.g. from vascular vasoconstriction or muscular contraction).

In this context, tissues formed of epithelial cells (e.g., cells lining the gut, blood vessels or ducts within organs) present particular problems for culturing on the lab-on-a-chip or organ-on-a-chip scale. This is in part because epithelial tissue is formed from cells which are precisely orientated and often vary at a sub-cellular level. For example, ciliated cells (such as those found in the digestive tract) present with microvilli on one side of the cell membrane (the apical surface which faces the external environment or lumen of the tissue) and smooth membranes on an opposite side (the basal surface).

To achieve this variation, it is necessary to establish chemical gradients localised to the cellular level, generally referred to as an apical-basal polarity. Maintaining the correct apical-basal polarity is essential for epithelial tissue form and function, as it determines the location of the functional membrane molecules such as adhesion molecules that hold the cells together laterally.

Although there has been some success at growing sheets of epithelial cells on top of membranes in 2D culture models (e.g., using apical-basal millicell culture plates), successfully growing 3D epithelial tissues (such as blood vessels, kidney nephrons and bile ducts) without a membrane remains a challenge in 3D culture models.

### SUMMARY

In an aspect of the disclosure a cell culture device is provided, the cell culture device comprising a well plate and a cover sheet. The well plate comprises at least one culture arrangement, the culture arrangement comprises a row of wells having a first outer well, a central well and a second outer well. An access port provides an opening into the first outer well. A passage (e.g., a straight or linear passage) extends from the access port to each of the first outer well, central well and second outer well. The passage passes through a first hole in a first well wall separating the first outer well and the central well. The passage passes through a second hole in a second well wall separating the central well and second outer well. The cover sheet is provided on an underside of the well plate.

The first well wall may comprise a first protrusion that protrudes into the central well. The second well wall may comprise a second protrusion that protrudes into the central well.

The first protrusion may extend around (e.g., circumscribes) the first hole. The second protrusion may extend around (e.g., circumscribes) the second hole. The passage may pass through the first protrusion and the second protrusion.

The first protrusion may comprise a first flange. The second protrusion may comprise a second flange. The first flange and/or the second flange may extend perpendicular to the passage (e.g., in a respective plane perpendicular to the passage). The first flange may extend around a perimeter of a distal end of the first protrusion. The second flange may extend around a perimeter of a distal end of the second protrusion.

The first protrusion may extend over less than 25% of a distance between the first well wall and the second well wall along an extension direction of the passage.

The second protrusion may extend over less than 25% of a distance between the first well wall and the second well wall along an extension direction of the passage.

One or more walls of the central well may comprise one or more anchorage channels.

The anchorage channels may include a generally horizontally section extending into a lateral wall of the central well. The anchorage channels may include a generally vertically extending section.

The cover sheet may comprise an optically transparent polymer. The optically transparent polymer may be a cyclo olefin polymer.

The well plate may comprise a polymer (e.g., a biocompatible polymer). The well plate may comprise a structure formed from a biocompatible resin (e.g., a photocurable resin).

The central well may comprise a rectangular cross-section. The first outer well may comprise a first circular cross-section. The second outer well may comprise a second circular cross-section.

The passage may have a diameter between 200 μm and 1 mm, between 300 μm and 600 μm, or of approximately 500 μm.

The well plate may comprise a plurality of culture arrangements in a parallel configuration. The plurality of culture arrangements may comprise six culture arrangements.

A cell culture matrix may be provided in the central well. The cell culture matrix may be a three-dimensional culture medium. The cell culture matrix may comprise one or more components (e.g. proteins) of an extracellular matrix (e.g. collagen, gelatin). The cell culture matrix may comprise collagen, gelatine and/or agar (e.g., nutrient agar or blood agar). The cell culture matrix may comprise bovine collagen and/or rat tail collagen. The cell culture matrix may comprise cells, e.g. immune cells. The cell culture matrix may extend from the first well wall to the second well wall. The passage may extend through a channel in the cell culture matrix. The channel may extend from the first hole to the second hole.

In another aspect of the disclosure, a method of growing cells to form a duct or vessel is provided. The method comprises: (i) providing a cell culture device comprising a well plate and a cover sheet, the well plate including a first outer well, a central well and a second outer well, an access port which provides an opening into the first outer well, a cell culture matrix provided in the central well, and a passage extending from the access port to each of the first outer well, central well and second outer well, the passage passing through a first hole in a first well wall which separates the first outer well and the central well and the passage passing through a second hole in a second well wall which separates the central well and second outer well, wherein the cell culture matrix extends from the first well wall to the second well wall and the passage extends through a channel in the cell culture matrix, wherein the cover sheet is provided on an underside of the well plate and wherein an end plug is connected to the access port to seal the access port; (ii) adding a cell culture medium comprising cells to at least one of the first outer well and the second outer well; (iii) incubating the cell culture device under conditions suitable to allow the cells to adhere to a wall of the channel; and (iv) incubating the cell culture device under conditions suitable to allow cells adhered to the wall of the channel to proliferate (e.g. to reach confluence).

The cells may comprise cholangiocytes, e.g. patient-derived cholangiocytes. The cholangiocytes may be obtained, isolated and propagated using suitable techniques known in the art (e.g. protocols described in Tysoe et al., 2019, Nature Protocols, 14, pp. 1884-1925, which is incorporated herein by reference). For example, the cells may be obtained from a subject using endoscopic retrograde cholangiopancreatography (ERCP).

Conditions suitable to allow the cells to adhere to the wall of the channel and/or proliferate may be readily determined by the skilled person. In a representative example, the conditions may include incubating the device at a predetermined temperature (e.g. 30-40° C., e.g. 37° C.) for a predetermined period of time (e.g. about 4-48 hours, such as about 8-32 or 12-24 hours). The device may be incubated under conditions that result in fluid flow within the channel, such as gravity-driven fluid flow, e.g. by placing the device on a moving platform.

The method may comprise growing a bile duct (e.g., a three dimensional culture model of a bile duct).

The method may include treating the cell culture device before adding the cell culture medium comprising cells to the outer wells. The treating may comprise the steps of: adding a basal culture medium to the wells; removing the basal culture medium from the wells; adding a cell culture medium to first outer well and the second outer well, and optionally the central well; and removing the cell culture medium from the first outer well and the second outer well. The method may include removing cell culture medium from the central well after adding the cell culture medium comprising cells to the first outer well and the second outer well and before incubating the cell culture device.

In another aspect of the disclosure a system comprising a well plate, cover sheet and an alignment insert is provided. The well plate comprises at least one culture arrangement, the culture arrangement comprising a row of three wells having a first outer well, a central well and a second outer well, an access port to provide an opening into the first outer well. A passage extends from the access port to each of the first outer well, central well and second outer well, the passage passing through a first hole in a first well wall separating the first outer well and the central well and the passage passing through a second hole in the second well wall separating the central well and second outer well. The cover sheet is provided on an underside of the well plate. The alignment insert comprises a plug and an alignment extension extending from the plug, and a guide channel extends from an insert end of the plug, through the plug and at least partly through the alignment extension. The plug provides an interface to the access port of the culture arrangement of the well plate, such that the guide channel aligns with the passage of the culture arrangement of the well plate.

The plug may be inserted into the access port.

The plug may comprise a frustoconical insert end portion. The guide channel may be positioned eccentrically from a centre of the insert end portion.

The well plate may comprise a plurality of culture arrangements. The alignment insert may comprise a plurality of plugs and alignment extensions.

The system may comprise a channel-forming apparatus having a cross-section equal to or smaller than each of: a cross-section of the first hole, and the second hole; a cross-section of the passage; and a cross-section of the guide channel. The channel-forming apparatus may comprise glass.

In another aspect of the disclosure, a method of fabrication of a cell culture device is provided. The method comprises forming a well plate by an additive manufacturing process, the well plate comprising a culture arrangement, the culture arrangement including a row of three wells having a first outer well, a central well and a second outer well. An access port provides an opening into the first outer well. A straight passage extends from the access port to each of the first outer well, central well and second outer well, the passage passing through a first hole in a first well wall which separates the first outer well and the central well and the passage passing through a second hole in a second well wall which separates the central well and second outer well.

The method may further include adhering a cover sheet to an underside of the well plate.

The method may further include sterilizing the well plate.

The method may further include: inserting a channel-forming apparatus into the passage so that it extends from the access port to the second outer well; introducing a cell culture matrix preparation (e.g., a matrix forming culture medium) to the central well; incubating the cell culture device and cell culture matrix preparation under conditions suitable to form a cell culture matrix in the central well; and removing the channel-forming apparatus to provide a channel in the cell culture matrix from the first hole in the first well wall to the second hole in the second well wall.

Inserting the channel-forming apparatus into the passage may comprise introducing an alignment insert into the access port and inserting the channel-forming apparatus through a guide channel within the alignment insert into the passage.

The alignment insert may comprise a plug and an alignment extension extending from the plug. The guide channel may extend from an insert end of the plug, through the plug and at least partly through the alignment extension.

The method may include introducing the alignment insert into the access port such that the guide channel aligns with the passage.

Forming the well plate by additive manufacturing may include forming the well plate as a mono-block.

The first well wall and the second well wall may each comprise a protrusion that protrudes into the central well. The protrusions may be produced by additive manufacturing simultaneously and integrally with the mono-block of the well plate.

Preferred embodiments will now be described, by way of example only, with reference to the drawings.

### BRIEF DESCRIPTION OF THE DRAWINGS

FIG. 1 shows an isometric view of an example cell culture device.

FIG. 2 shows sectional view of the example cell culture device of FIG. 1.

FIG. 3 shows an isometric view of various anchoring features for cell culture matrix adhesion.

FIGS. 4A-4D show a top view of a culture arrangement and various stages in the formation of a channel through a cell culture matrix.

FIGS. 5A-5C show a channel forming system including a plurality of alignment inserts.

FIG. 6 shows a method of culturing cells.

FIG. 7 shows a sectional view of a cell culture device.

FIGS. 8A-8C show a top view, underside view and end on view respectively of an example cell culture device.

### DETAILED DESCRIPTION

A culture arrangement (e.g., a cell culture chip, device or system) as described below can, for example, be used to grow any manner of epithelial tissue and is particularly well suited for growing epithelial tissue having tubular configurations (e.g., blood vessels, bile ducts, nephrons and the like).

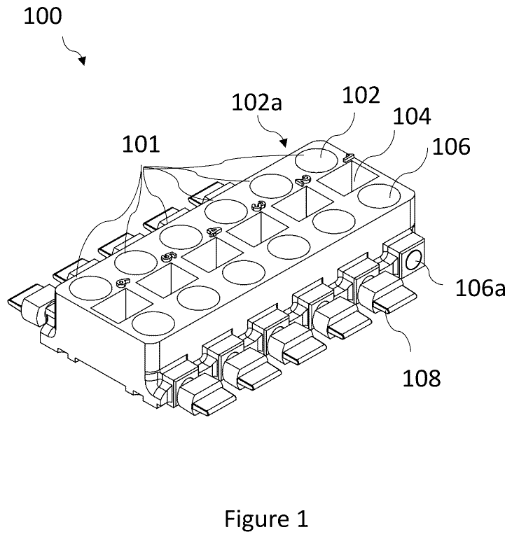

In an example (e.g., as shown in FIG. 1), a cell culture device comprises a well plate 100. The well plate 100 comprises a plurality of culture arrangements 101 (of which six are shown). Each of the culture arrangements 101 is positioned parallel to the next. The spacing between adjacent culture arrangements 101 may be equal to that of a standard 384-well microplate (i.e., 4.5 mm), or that of a standard 96-well microplate (i.e. 9 mm). A culture arrangement 101 comprises a central well 104 positioned between a first outer well 102 and a second outer well 104. The first outer well 102, central well 104 and second outer well 106 are configured in a straight line to form a row of wells. The central well 104 comprises a rectangular cross-section, the first outer well 102 comprises a first circular cross-section and the second outer well 106 comprises a second circular cross-section.

A first access port 102a is provided in-line with the row of wells adjacent the first outer well 102. A second access port 106a is provided in-line with the row of wells adjacent the second outer well 106. In other examples (not shown), the second access port 106a may be omitted. The first access port 102a and/or the second access port 106a may comprise a tapered or frustoconical surface. The first access port 102a and/or the second access port 106a may be configured for connection to off-chip equipment such as a syringe or other microfluidic pump. The first access port 102a and/or the second access port 106a may comprise standardised fittings such a luer fitting or a mini luer fitting.

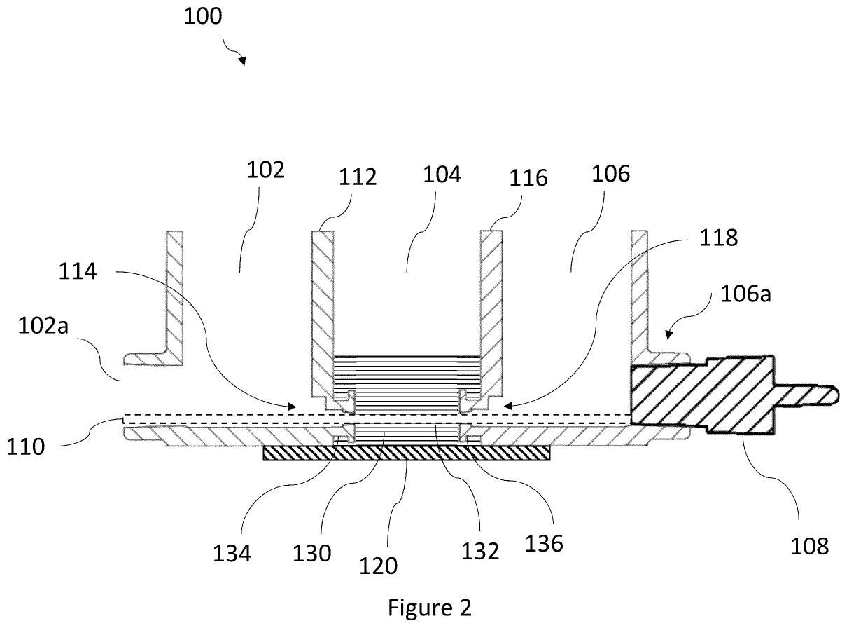

With further reference to FIG. 2, a passage 110 (e.g., a continuous, straight or linear passage) is provided extending from the first access port 102a to and through each of the first outer well 102, central well 104 and second outer well 106. In the example shown in FIG. 2, an end plug 108 (e.g., a silicone end plug) is provided in the second access port 106a such that the passage 110 terminates in the second outer well 106 (this will also be the case in embodiments where the second access port 106a is omitted). The end plug 108 may be a mini luer connector. Another end plug 108 may be provided in the first access port 102 to seal the well plate 100 during use. The end plugs 108 may each be formed from resilient material and include a frustoconical surface arranged to interface with and seal the first access port 102a or the second access port 106a respectively. The passage 110 passes through a first hole 114 in a first well wall 112 which separates the first outer well 102 and the central well 104, and passes through a second hole 118 in a second well wall 116 which separates the central well 104 and second outer well 106. The passage 110 may have a diameter between 200 μm and 1 mm, between 300 μm and 600 μm or of approximately 500 μm.

The well plate 100 includes a respective opening above (i.e., on the top side) each of the first outer well 102, the central well 104 and the second outer well 106. In the illustrated example, an opening is also provided below (i.e., on the bottom or underside) the central well 104. That is, each of the first outer well 102 and the second outer well 106 have a closed base, whereas the central well 104 has an open base. A cover sheet 120 (e.g., an optically transparent cover sheet such as a slide or coverslip) is provided on the underside of the well plate 100. The cover sheet 120 provides an optically transparent viewing window for monitoring cell growth in the central well 104. The cover sheet 120 which provides closure to the underside or bottom end of the central well 104 is removably attached to the well plate 100. The well plate 100 may comprise or otherwise be formed from a polymer such as a photopolymer resin or biocompatible resin. The well plate 100 may be formed by an additive manufacturing process such as by a UV Curable 3D printing process. The cover sheet 120 may comprise or otherwise be formed from an optically transparent polymer such as a cyclo olefin polymer. The cover sheet 120 may be adhered to the underside of the well plate 100. For example, the cover sheet 120 may be attached to a well plate formed of resin by means of the resin from which it is formed. Alternatively, the cover sheet 120 retained to the underside by mechanical means (not shown). Forming the well plate 100 and cover sheet 120 from polymer materials having similar thermal expansion properties ensures structural integrity during autoclaving. Alternatively, a glass cover sheet 120 can be used.

In other examples (not shown), a viewing window may be integral to the well plate 100. That is, the underside of the well plate may be formed so as to be optically transparent (e.g., formed from optically transparent materials such as glass or high-density polycarbonate).

The well plate 100 and culture arrangement 101 may be prepared (e.g., pre-prepared) with a cell culture matrix 130 disposed in the central well 104. The cell culture matrix may be a three-dimensional culture medium. The cell culture matrix may comprise one or more components (e.g. proteins) of an extracellular matrix (e.g. collagen, gelatin). The cell culture matrix may 130 comprise collagen, gelatin or agar. The cell culture matrix 130 may comprise bovine collagen and/or rat tail collagen. With continued reference to FIG. 2, the cell culture matrix 130 is provided with a channel 132 extending therethrough such that the passage 110 passes through the channel 132 as it extends from the first outer well 102 to the second outer well 106. In order to improve adhesion of the cell culture matrix 130 to the central well 104, one or more anchoring features may be provided. To improve visibility, the channel 132 is preferably arranged close to the cover sheet 120 or window. Consequently, the passage 110 may be arranged close to the underside of the cell culture device. In the illustrated examples, the passage 110 is eccentric to a centre of the first access port 102a and/or the second access port 106a.

In the example shown in FIG. 2, a first protrusion 134 is provided extending or protruding from the first well wall 112 into the central well 104 and a second protrusion 136 is provided extending or protruding from the second well wall 116 into the central well 104. The first protrusion 134 extends around (e.g., circumscribes) the first hole 114. The second protrusion 136 extends around (e.g., circumscribes) the second hole 118. The passage 110 passes through the first protrusion 134 and the second protrusion 136. In the illustrated example, the first protrusion 134 comprises a first flange and the second protrusion 136 comprises a second flange. The first flange extends circumferentially (e.g., around a perimeter) from a distal end of the first protrusion 134 in a plane (e.g., a first plane) which is perpendicular to the passage 110. Similarly, the second flange extends circumferentially (e.g., around a perimeter) from a distal end of the second protrusion 136 in a plane (e.g., a second plane) which is perpendicular to the passage 110. The first protrusion 134 may extend over less than 25% of a distance between the first well wall 112 and the second well wall 116 along an extension direction of the passage 110. The second protrusion 136 may extend over less than 25% of the distance between the first well wall 112 and the second well wall 116 along the extension direction of the passage 110 (e.g., in the opposite direction to the first protrusion 134).

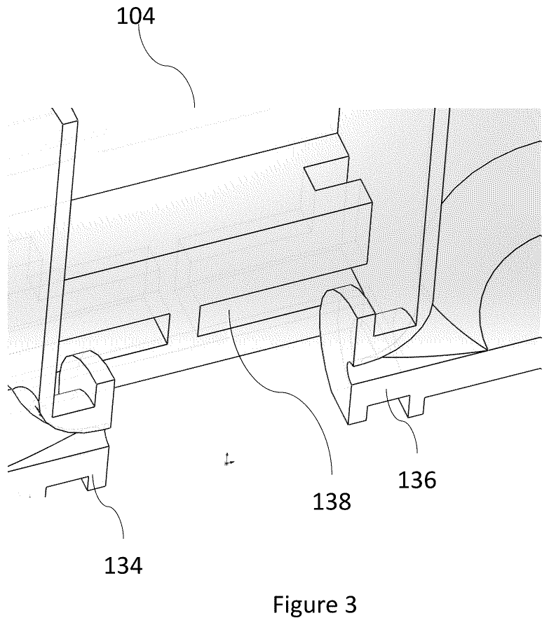

The one or more anchoring features may additionally or alternatively include one or more features to anchor the cell culture matrix 130 to one or more walls of the central well 104 (e.g., lateral walls between adjacent culture arrangements 101). In the example shown in FIG. 3, anchorage channels 138 are provided (e.g., formed into) the lateral walls of the central well 104. When a matrix-forming cell culture medium (e.g., liquid) is disposed in the central well 104, the matrix-forming cell culture medium flows into the anchorage channels 138. Once set the matrix-forming cell culture medium forms the cell culture matrix 130 extending into the anchorage channels 138 preventing detachment or warping of the cell culture matrix 130. The anchorage channel 138 may extend generally horizontally into a lateral wall of the central well 104. The anchorage channel 138 may include one or more vertically extending vents. The vents allow air bubbles to escape the anchorage channels 138 so that the cell culture matrix 130 can fill the anchorage channels 138.

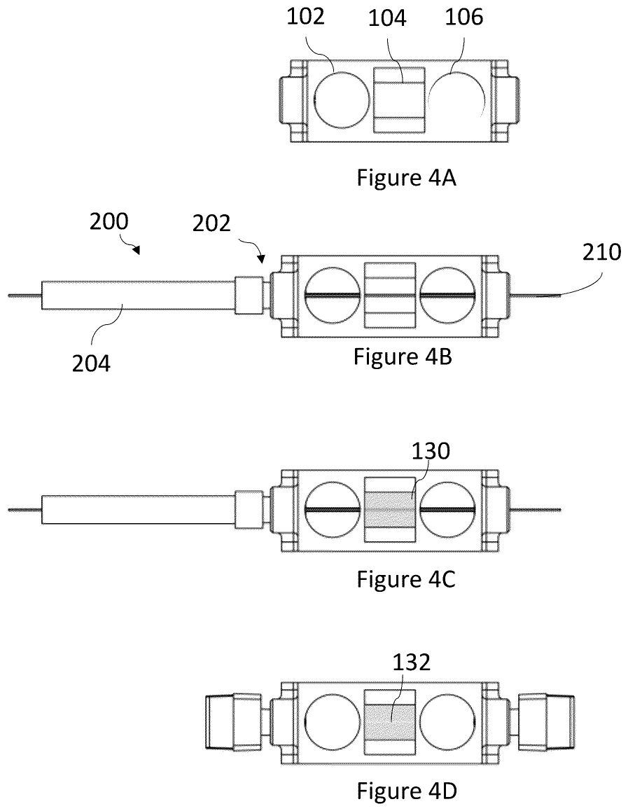

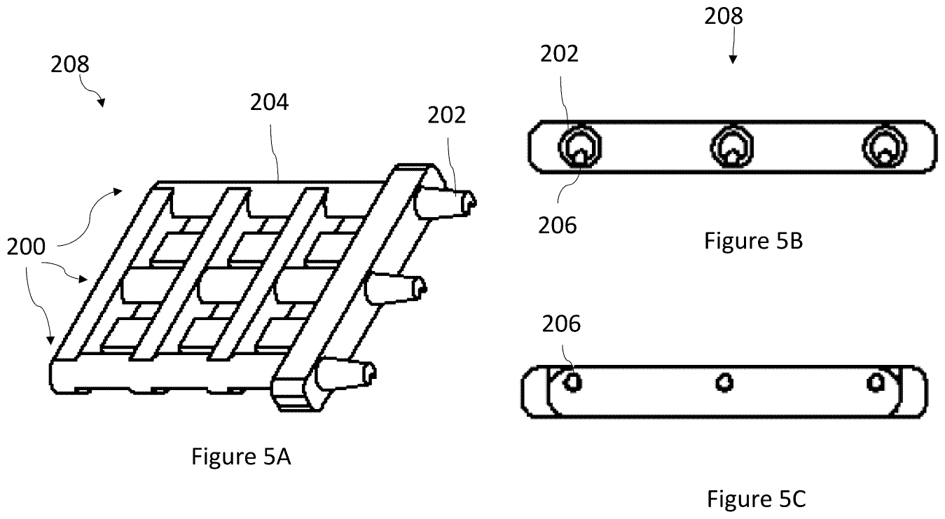

With reference to FIGS. 4A-4D, a system is provided to form the channel 132 within the cell culture matrix 130. In the example shown, the channel forming system includes an alignment insert 200 and a channel forming apparatus 210. The alignment insert 200 comprises a plug 202 and an alignment extension 204 extending from the plug 202. The plug 202 is configured to provide an interface with the access ports 102a, 106a (e.g., the plug 202 is shaped to be complimentary to the access ports 102a, 106a). In the example shown, the plug 202 comprises a frustoconical surface (e.g., a frustoconical insert end portion). With further reference to FIGS. 5A-5C, a guide channel 206 extends from an insert end of the plug 202, through the plug 202 and at least partly through the alignment extension 204. The guide channel 206 is arranged to align with the passage 110 of the culture arrangement 100. In the illustrated example, the guide channel 206 is positioned eccentrically from a centre of the plug 202 (e.g., eccentrically from a centre of the insert end portion). The alignment insert 200 may comprise one or more alignment features for ensuring the eccentrically positioned guide channel 206 is rotationally aligned with the eccentrically positioned passage 110.

The channel forming apparatus 210 comprises a cross-section equal to or smaller than each of a cross section of the first hole 114, the second hole 118 and the guide channel 206. The channel forming apparatus 210 may comprise an elongate rod, a capillary, a needle or a wire. Preferably, the channel forming apparatus 210 comprises glass (e.g., an elongate glass rod or a glass capillary). It has been found that a glass channel forming apparatus 210 reduces the likelihood of air bubble nucleation in the subsequently formed channel 132.

The channel forming system may be used to form the channel 132 in the cell culture matrix 130. With reference to FIGS. 4A-4D a method for forming the channel 132 includes inserting the alignment insert 200 into the first access port 102a (or equally, into the second access port 106a). The channel-forming apparatus 210 is subsequently inserted into the passage 110 via the guide channel 206 so that the channel-forming apparatus 210 extends from the first access port 102a to at least the second outer well 106 or out of the second access port 106a (see FIG. 4B). The matrix-forming cell culture medium is then introduced to the centre well 104 and allowed to set (e.g., cure by incubation under conditions suitable to form the cell culture matrix 130) with the channel-forming apparatus 210 held in place (see FIG. 4C). Once the matrix-forming cell culture medium is set into the cell culture matrix 130, the channel-forming apparatus 210 is removed to provide the channel 132 in the cell culture matrix 130 (see FIG. 4D). Each of the first access port 102a and/or the second access port 106a may then be sealed with one or more end plugs 108.

In the example shown in FIGS. 5A-5C, the channel forming system includes a plurality of alignment inserts 200 on an alignment frame 208. The plurality of alignment inserts 200 having a corresponding plurality of guide channels 206 serve as a co-alignment feature to ensure that all plurality of guide channels 206 are simultaneously rotationally aligned with a corresponding plurality of passages 110 in the plurality of culture arrangements 101. In the illustrated example, the well plate 100 includes six culture arrangements and the alignment frame 208 includes three alignments inserts 200, however, any number of inserts are possible. For example, the alignment frame 208 may have a matching number of inserts to a number of culture arrangements (e.g., six), such that all the channels 132 may be formed simultaneously. Alternatively, the alignment frame 208 may have a number of alignment inserts 200 a fraction of the number of culture arrangements 101 such that the channels 132 may be formed in predefined batches.

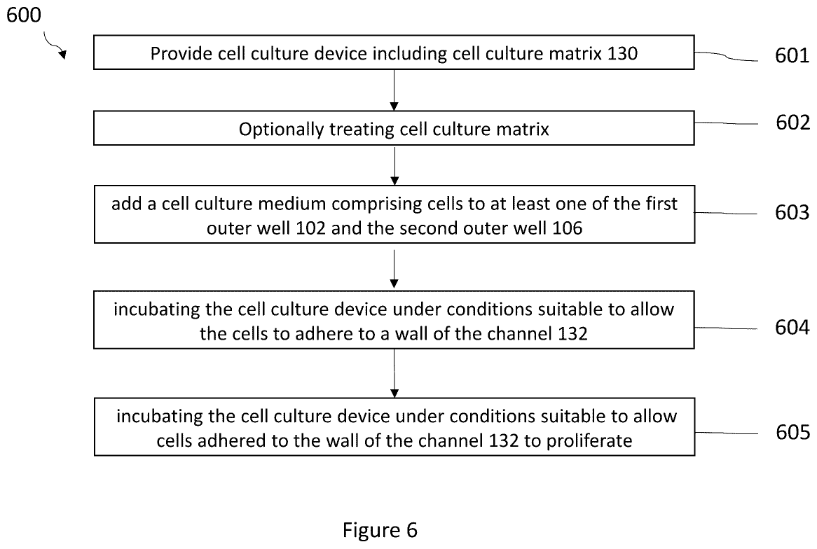

The cell culture device may be used to grow cells (e.g., epithelial cells) to form a duct or vessel. In one example, a method 600 of growing cells to form a duct or vessel includes the steps outlined in FIG. 6. The method 600 may comprise growing a bile duct.

Step 601 includes providing a cell culture device including a cell culture matrix 130. The cell culture device comprising a well plate and a cover sheet, the well plate including a first outer well, a central well and a second outer well, an access port which provides an opening into the first outer well, a cell culture matrix provided in the central well, and a passage extending from the access port to each of the first outer well, central well and second outer well, the passage passing through a first hole in a first well wall which separates the first outer well and the central well and the passage passing through a second hole in a second well wall which separates the central well and second outer well, wherein the cell culture matrix extends from the first well wall to the second well wall and the passage extends through a channel in the cell culture matrix, wherein the cover sheet is provided on an underside of the well plate and wherein an end plug is connected to the access port to seal the access port (e.g., as described above).

Step 603 includes adding a cell culture medium comprising cells (e.g., a suspension of epithelial cells, e.g. choliangiocytes) to at least one of the first outer well 102 and the second outer well 106 and allowing the cell culture medium comprising cells to infiltrate the channel 132. Step 603 may optionally include aspirating (e.g., drawing) the cell culture medium comprising cells between the first outer well 102 and the second outer well 106 so as to fill the channel 132 with the cell culture medium comprising cells and remove any air bubbles from the channel 132. Alternatively, the cell culture medium comprising cells may be allowed to passively fill the channel 132 (e.g., under the action of gravity or capillary forces). Step 603 may optionally include removing the cell culture medium comprising cells from the at least one of the first outer well 102 and the second outer well 106 (e.g., once the channel has been filled with the culture medium comprising cells). The cell culture medium comprising cells may comprise cholangiocytes isolated from bile ducts during endoscopic retrograde cholangiopancreatography.

Step 604 includes incubating the cell culture device under conditions suitable to allow the cells to adhere to a wall of the channel (e.g., for a predetermined time at a predetermined temperature). Step 604 may optionally include continuously perfusing cell culture medium comprising cells from the first access port 102a to the second access port 106a (e.g., with one or more microfluidic pumps connected to the access ports 102a 106a). Step 604 may optionally include removing the cell culture medium comprising cells from the wells, and optionally the channel, and providing a cell culture medium (e.g., a fresh cell culture medium or growth medium without a cell suspension).

Step 605 includes incubating the cell culture device under conditions suitable to allow cells adhered to the wall of the channel to proliferate (e.g., for a second predetermined time at a second predetermined temperature). Step 605 may optionally include periodically replacing the cell culture medium (e.g., growth medium) with fresh cell culture medium. Step 605 may optionally include continuously perfusing cell culture medium (e.g., growth medium) from the first access port 102a to the second access port 106a (e.g., with one or more microfluidic pumps connected to the access ports 102a 106a).

The method 600 may further comprise a step 602 of treating (e.g., pre-treating) the cell culture matrix 130 to establish a basal-apical polarity (e.g., a chemical or pH gradient). For example, step 602 may include adding a basal culture medium to the central well 104 and allowing the basal culture medium to permeate the cell culture matrix 130 before the step of adding the cell culture medium comprising cells to at least one of the first outer well 102 and the second outer well 106. The basal culture medium may subsequently be removed from the central well. Step 602 may optionally include adding the basal culture medium to at least one of the first outer well 102 and the second outer well 106 and allowing the basal culture medium to infiltrate the channel 132 before subsequently removing the basal culture medium from the at least one of the first outer well 102 and the second outer well 106 and the channel 132. Step 602 may include adding a cell culture medium (e.g., a culture medium comprising cytokines) to at least one of the first outer well 102, the central well 104 and the second outer well 106 and allowing the cell culture medium to permeate the cell culture matrix 130 and/or infiltrate the channel 132 before subsequently removing the cell culture medium (e.g., comprising cytokines) from the at least one of the first outer well 102, the central well 104 and the second outer well 106 and/or the channel 132. Step 602 may include adding the cell culture medium (e.g., comprising cytokines) to the central well 104, and step 603 may include removing the cell culture medium (e.g., comprising cytokines) from the central well 104 after adding the cell culture medium comprising cells to at least one of the first outer well 102 and the second outer well 106.

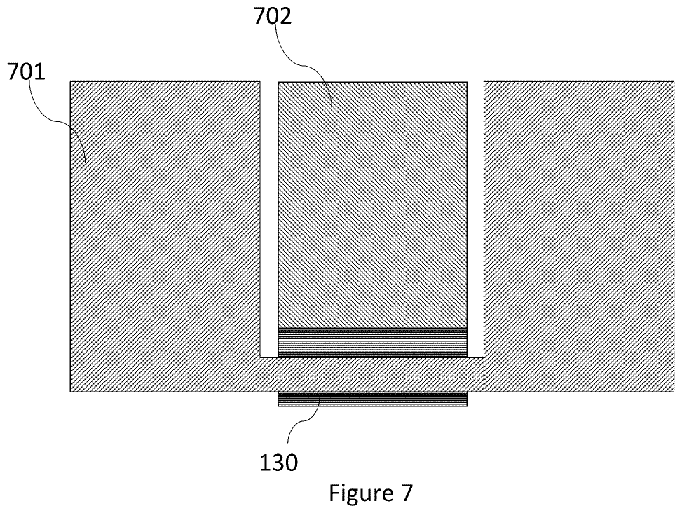

In this configuration, the first outer well 102, the second outer well 106 and the channel 132 collectively form an apical region 701, while the central well 104 and bulk of the cell culture matrix 130 form a basal region 702 (e.g., as shown schematically in FIG. 7). The first outer well 102 and the second outer well 104 may respectively be referred to as the first apical well and the second apical well, while the central well 104 may be referred to as the basal well.

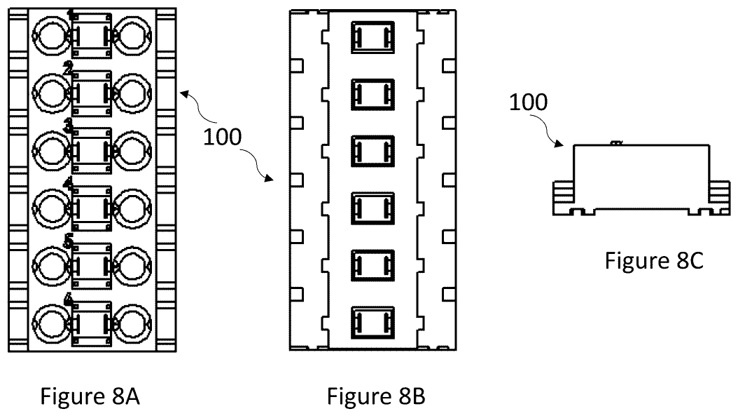

FIGS. 8A to 8C show a plan view, an underside view and an end view respectively of an example of a well plate 100 for a cell culture device as described above with regard to FIG. 1 (the same reference numerals apply). The disclosure provides an in vitro platform modelling the human biliary system including its surrounding liver tissue using “organ on a chip” technology. The platform offers the possibility of combining patient-derived biliary cells with their surrounding liver tissue in an in vivo like architecture and allows for flexibility to study further factors in disease progression such as the immune response.

The interaction between the immune system and the environment is believed to be the driving force behind many hepatic inflammatory diseases. The biliary system is exposed to antigens that can potentially trigger the immune system from two different sources: the bile and the blood draining through the portal circulation to the liver. Using a functional “bile duct chip” would give the opportunity for direct investigation of the interaction of biliary cells and immune components in a highly manipulable system that can generate new insights into the pathology of cholangiopathies, leading to a possible new platform for developing therapeutics for a variety of hepatobiliary diseases including PSC.

The system includes aspects of the immune system, the epithelial barrier, and the biliary microenvironment in integrated and completely novel manner. This provides significant advances over the currently used methods in the field as well having greater physiological relevance than other systems in development, which lack primary human biological material, use undesirable materials and have limited scalability.

We claim:

1. A cell culture device comprising a well plate and a cover sheet,
the well plate comprising:
a culture arrangement, the culture arrangement comprises a row of wells having a first outer well, a central well and a second outer well;
an access port which provides an opening into the first outer well; and
a passage extending from the access port to each of the first outer well, central well and second outer well, the passage passing through a first hole in a first well wall separating the first outer well and the central well and the passage passing through a second hole in a second well wall separating the central well and second outer well,
wherein the cover sheet is provided on an underside of the well plate.

2. The cell culture device as claimed in claim 1, wherein the first well wall comprises a first protrusion that protrudes into the central well and the second well wall comprises a second protrusion that protrudes into the central well.

3. The cell culture device as claimed in claim 2, wherein the first protrusion extends around the first hole, the second protrusion extends around the second hole, and the passage passes through the first protrusion and the second protrusion.

4. The cell culture device as claimed in claim 3, wherein the first protrusion comprises a first flange and the second protrusion comprises a second flange, wherein, optionally, the first flange and the second flange extend perpendicular to the passage, wherein, optionally, the first flange extends around a perimeter of a distal end of the first protrusion.

5. The cell culture device as claimed in claim 2, wherein the first protrusion extends over less than 25% of a distance between the first well wall and the second well wall along an extension direction of the passage.

6. The cell culture device as claimed in claim 1, wherein one or more walls of the central well comprise one or more anchorage channels.

7. The cell culture device as claimed in claim 1, wherein the cover sheet comprises an optically transparent polymer, wherein, optionally, the optically transparent polymer is a cyclo olefin polymer.

8. The cell culture device as claimed in claim 1, wherein the well plate comprises a structure formed from a biocompatible resin.

9. The cell culture device as claimed in claim 1, wherein the central well comprises a rectangular cross-section, the first outer well comprises a first circular cross-section and the second outer well comprises a second circular cross-section.

10. The cell culture device as claimed in claim 1, wherein the passage has a diameter between 200 μm and 1 mm, wherein, optionally, the diameter is between 300 μm and 600 μm or approximately 500 μm.

11. The cell culture device as claimed in claim 1, wherein the well plate comprises a plurality of culture arrangements in a parallel configuration, wherein, preferably, the plurality of culture arrangements comprises six culture arrangements.

12. The cell culture device of claim 1, comprising a cell culture matrix provided in the central well, wherein the cell culture matrix extends from the first well wall to the second well wall, and
the passage extends through a channel in the cell culture matrix, wherein the channel extends from the first hole to the second hole.

13. A method of growing cells to form a duct or vessel, the method comprising:
(i) providing a cell culture device comprising a well plate and a cover sheet, the well plate including a first outer well, a central well and a second outer well, an access port which provides an opening into the first outer well, a cell culture matrix provided in the central well, and a passage extending from the access port to each of the first outer well, central well and second outer well, the passage passing through a first hole in a first well wall which separates the first outer well and the central well and the passage passing through a second hole in a second well wall which separates the central well and second outer well, wherein the cell culture matrix extends from the first well wall to the second well wall and the passage extends through a channel in the cell culture matrix, wherein the cover sheet is provided on an underside of the well plate and wherein an end plug is connected to the access port to seal the access port;
(ii) adding a cell culture medium comprising cells to at least one of the first outer well and the second outer well;
(iii) incubating the cell culture device under conditions suitable to allow the cells to adhere to a wall of the channel; and
(iv) incubating the cell culture device under conditions suitable to allow cells adhered to the wall of the channel to proliferate.

14. The method as claimed in claim 13, wherein before adding the cell culture medium comprising cells to the outer wells, the cell culture device is treated according to the following steps:
a basal culture medium is added to the wells;
the basal culture medium is removed from the wells;
a cell culture medium is added to first outer well and the second outer well and optionally the central well; and
the cell culture medium is removed from the first outer well and the second outer well, and
wherein, after adding the cell culture medium comprising cells to the first outer well and the second outer well, any cell culture medium in the central well is removed before incubating the cell culture device.

15. A system comprising a well plate, cover sheet and an alignment insert,
the well plate comprising at least one culture arrangement, the culture arrangement comprising a row of three wells having a first outer well, a central well and a second outer well, an access port to provide an opening into the first outer well; a passage extending from the access port to each of the first outer well, central well and second outer well, the passage passing through a first hole in a first well wall separating the first outer well and the central well and the passage passing through a second hole in the second well wall separating the central well and second outer well;
wherein the cover sheet is provided on an underside of the well plate;
wherein the alignment insert comprises a plug and an alignment extension extending from the plug, and a guide channel extends from an insert end of the plug, through the plug and at least partly through the alignment extension,
wherein the plug provides an interface to the access port of the culture arrangement of the well plate, such that the guide channel aligns with the passage of the culture arrangement of the well plate.

16. The system as claimed in claim 15, wherein the plug is inserted into the access port.

17. The system as claimed in claim 15, wherein the plug comprises a frustoconical insert end portion and the guide channel is positioned eccentrically from a centre of the insert end portion.

18. The system of claim 15, wherein the well plate comprises a plurality of culture arrangements and the alignment insert comprises a plurality of plugs and alignment extensions.

19. The system as claimed in claim 15, comprising a channel-forming apparatus having a cross-section equal to or smaller than each of: a cross-section of the first hole, and the second hole; a cross-section of the passage; and a cross-section of the guide channel.

20. The system as claimed in claim 19, wherein the channel-forming apparatus comprises glass.
